(19) United States
(12) Patent Application Publication
(10) Pub. No.: US 2020/0354710 A1
(43) Pub. Date: Nov. 12, 2020

(54) MONITORING ACTIVATION OF STEM CELLS

(71) Applicant: Clear LLC, Mill Valley, CA (US)

(72) Inventor: Beth McDougall, Mill Valley, CA (US)

(21) Appl. No.: 16/871,307

(22) Filed: May 11, 2020

Related U.S. Application Data
(60) Provisional application No. 62/846,537, filed on May 10, 2019.

(51) Int. Cl. C12N 13/00; A61N 5/06; A61K 35/28

(52) U.S. Cl. CPC C12N 13/00; A61N 2005/0662; A61K 35/28; A61N 5/062

(57) ABSTRACT

Activation of stem cell activation is monitored and customized based on a patient's skin condition, including skin coloration, texture, thickness, and pigmentation. Skin properties are continuously monitored during stem cell activation, including moisture, permeability, temperature, blood circulation, conductivity, blood pressure, and metabolism. Energy used for stem cell activation can be adjusted in real-time based on changes in the patient's skin condition or properties. Monitoring stem cell activation can also include measuring a molecule that is associated with stem cell activation, skin damage, or reactive oxygen species generation.

### PRIORITY INFORMATION

This application claims the benefit of priority to U.S. Provisional Patent Application No. 62/846,537, filed on May 10, 2019, which is incorporated herein by reference in its entirety.

### FIELD OF THE INVENTION

The field of the invention is monitoring activation of stem cells.

### BACKGROUND

The following description includes information that may be useful in understanding the present invention. It is not an admission that any of the information provided herein is prior art or relevant to the presently claimed invention, or that any publication specifically or implicitly referenced is prior art.

Stem cell activation usually involves using an energy source, for example, laser or plasma. Ex vivo activation is where blood is removed from the body, plasma fraction is extracted from the blood, and then the dormant stem cells in the plasma fraction are activated. For example, United States Patent Publication US20180280509A1 by Schena teaches treating blood with non-thermal plasma generated at atmospheric pressure and room temperature in vitro, where 8 J/cm² is determine to be an energy threshold for cell damage. However, it does not teach how much energy is required to sufficiently activate stem cells. Similarly, U.S. Pat. Nos. 9,999,785 and 10,202,598 to Todd Ovokaitys teach using a laser beam to activate autologous or exogenous stem cells, but does not teach how much energy should be used.

The situation is more complicated under in vivo conditions, because energy needs to penetrate the skin, and it is difficult to quantify how much energy should be transdermally given to a patient, since different people have different skin conditions including coloration. U.S. Pat. No. 9,907,975 to Porter teaches using transdermal exposure to laser to activate mesenchymal stem cells, where the amount of energy delivered is greater than 5 Joules/cm² or least 1000 total Joules. However, Porter fails account for a patient's skin condition, since patients having a darker skin tone or thicker skin should be given more energy. Porter also fails to teach a safety mechanism to prevent a patient from being injured by overexposure.

In certain conditions, only a specific type of stem cells is preferably activated. For example, very small embryonic-like stem cells (VSELs) are much smaller than other types of stem cells. A protocol developed for activating other types of stem cells would not be suitable for activation of VSELs.

Thus, there is still a need for a method of monitoring activation of stem cells including VSELs, so that sufficient energy can be delivered safely to activate stem cells.

All publications identified herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

### SUMMARY OF THE INVENTION

The inventive subject matter provides apparatus, systems and methods in which activation of VSELs or other stem cells is monitored and customized based on a patient's skin condition.

Preferred methods of monitoring stem cell activation measure one or more skin conditions before delivering energy to the patient's skin. Different types of energy contemplated herein include light (including laser), heat, electric field, plasma radiation, acoustic waves (including ultrasound), and electromagnetic field (e.g., nuclear magnetic resonance (NMR)). The amount of energy given, including the intensity of the energy and duration of treatment, can be calculated based on empirical correlations between a condition of the skin and the amount of energy required to sufficiently activate stem cells (including very small embryonic-like stem cells (VSELs)) without damaging the stem cells or skin tissue. The calculation can also adjust for a variety of skin conditions, including coloration, skin texture, thickness, and pigmentation, etc. The condition of the skin can be measured in any suitable manner, but preferably noninvasively, for example, by a wearable skin monitor, a light sensor, etc. A light sensor can measure energy reflected from the skin and compare against a reference surface. Color coded discs can be used to determine suitable energy levels. Infrared (IR) or multi-channel light emitters can be used to determine absorption rate.

The skin conditions are continuously or periodically monitored during the treatment, to detect any changes in the skin conditions. These conditions include moisture, permeability, temperature, blood circulation in skin tissue, skin conductivity, cutaneous blood pressure, nerve activity in the skin (e.g., discharge rate, a nerve conductivity), and skin metabolism/metabolites, etc. The conditions of the skin can be measured in any suitable manner, but preferably noninvasively, for example, by a wearable skin monitor, an electrode contact with the skin, an electronic skin patch, or a chemical sensor measuring skin metabolites. In preferred embodiments, IR blood flow or temperature change via LED color reference can be used to monitor skin conditions. In some embodiments, the patient's skin is treated with a moisture soaking solution with 4-5% saline, about 95% water, and about 0.2 to 1% carboxymethylcellulose to produce a wet skin prune effect, which is a desired outcome. This treatment will increase the skin's permeability which will facilitate energy absorption and penetration.

Treatment is paused or terminated when the skin property reaches a predetermined threshold value. For example, the treatment is terminated or paused when temperature of the treated skin reaches 45° C., so that the skin is not overheated or burned. The treatment is resumed when the temperature of the treated skin drops below 40° C. Treatment is terminated when the desired amount of energy is delivered.

In some embodiments, the patient's skin is stimulated (e.g., with massage and/or heat) before treatment to increase blood flow to the skin tissue, and thus the number of stem cells in the local area. Moreover, skin can be bleached with a chemical to increase the amount of light that can penetrate the skin and reach the stem cells in the blood vessels. Moreover, microporation in the skin (e.g., using microneedles) can also be used to increase the light that can travel through the skin. Menthol can be used as a vasodilator. Topical organic Methyl salicylate or Methyl-Salicylate-Oil-of-Wintergreen-Wintergreen-Oil can also be used.

In some embodiments, the methods of monitoring stem cell activation include noninvasive characterization of a biomolecule of the human body. In preferred embodiments, the biomolecule is a biomarker of stem cell activation, or a biomarker of skin damage (e.g., reactive oxygen species). Such noninvasive characterization can be performed using a Fourier-Transform Infrared Spectroscopy (FTIR) or Nuclear Magnetic Resonance (NMR). Preferably, the biomolecule is in a body fluid, for example, blood, saliva or tear fluid. Alternate methodologies include using a dual channel asymmetrical laser to determine skin response adjacent to treatment location to analyze combined energy adsorption.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

FIG. 1 is a flowchart showing steps of monitoring stem cell activation in a patient, according to one embodiment of the inventive subject matter.

FIG. 2 is a flowchart showing steps of monitoring stem cell activation in a patient, according to another embodiment of the inventive subject matter.

### DETAILED DESCRIPTION

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Unless the context dictates the contrary, all ranges set forth herein should be interpreted as being inclusive of their endpoints, and open-ended ranges should be interpreted to include only commercially practical values. Similarly, all lists of values should be considered as inclusive of intermediate values unless the context indicates the contrary.

The recitation of ranges of values herein is merely intended to serve as a shorthand method of referring individually to each separate value falling within the range. Unless otherwise indicated herein, each individual value with a range is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

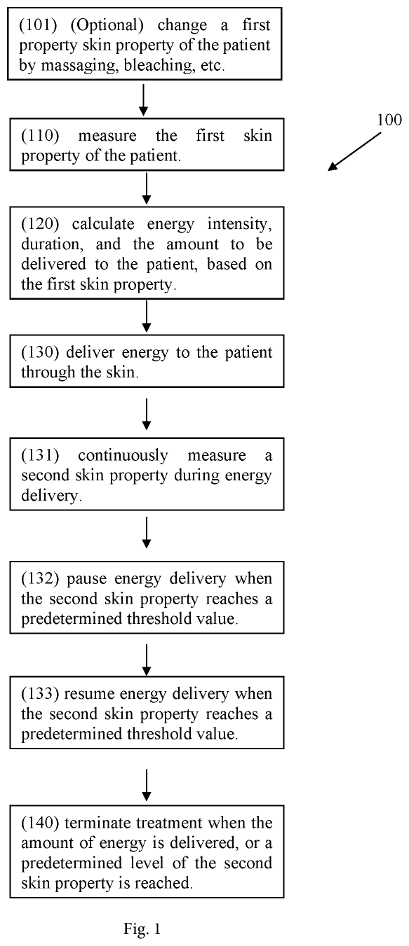

FIG. 1 is a flowchart 100 showing steps of monitoring stem cell activation in a patient. In optional step 101, a first skin property of the patent is changed by stimulation (e.g., with massage and/or heat) before treatment to increase blood flow to the skin tissue, and thus the number of stem cells in the local area. In some embodiments, a first skin property can be changed by bleaching with a chemical to increase the amount of light that can penetrate the skin and reach the stem cells in the blood vessels. In some embodiments, a first skin property can be changed by microporation in the skin (e.g., using microneedles) to increase the light that can travel through the skin. In some embodiments, a first skin property can be changed by topically applying menthol as a vasodilator, or organic Methyl salicylate, or Methyl-Salicylate-Oil-of-Wintergreen-Wintergreen-Oil.

In Step (110), a first skin property of the patient is measured, preferably using a non-invasive method, for example, by characterizing light reflection from the patient's skin. In preferred embodiments, one or more of the following skin properties can be measured: skin coloration, skin texture, thickness, and pigmentation.

In Step (120), the amount of energy (including energy intensity and duration) to be delivered to the patient is calculated based on the first skin property measured in step 110. The amount of energy given can be calculated based on empirical correlations between a skin property and the amount of energy required to sufficiently activate stem cells without damaging the stem cells or the skin tissue. The calculation can also adjust for a variety of skin conditions that may change the absorption rate of skin, including coloration, skin texture, thickness, pigmentation, scaring, etc. It is contemplated that a skin type that has a high absorption rate (e.g., light skin color, thin skin) is given less energy with lower intensity than a skin type that has a low absorption rate (e.g., dark skin color, thick skin).

In Step (130), energy is delivered to the patient through the skin based on the calculation in step 120, including the intensity of the energy, the duration of energy delivery, and the total amount of energy to be delivered. Different types of energy contemplated herein include light (including infrared and laser), heat, electric field, plasma radiation, acoustic waves (including ultrasound), and electromagnetic field (e.g., nuclear magnetic resonance (NMR)). Preferably, the duration of energy delivery is between 30 minutes and an hour. More preferably, energy is delivered in multiple sets (e.g., 3 to 5 sets), with each set between 5 to 15 minutes, having 30 seconds to 1 minute break between each set.

In Step (131), the second skin property is monitored, preferably continuously, during energy delivery. The second skin property to be monitored can be one or more of the following: moisture, permeability, temperature, blood circulation in skin tissue, skin conductivity, cutaneous blood pressure, skin metabolism, and a nerve activity of the patient's body. The nerve activity can be neuron discharge rate or a nerve conductivity monitored by a nerve conduction velocity (NCV) test, to assess any nerve damage and dysfunction caused by the energy delivery. The data related to the nerve activity can be transmitted to a processor, and the energy delivery can be adjusted accordingly, including decreasing or increasing the energy intensity, or pausing energy delivery. Preferably, energy delivery (intensity or the amount) is adjusted in real-time based on a change in the second skin property. As used herein, “real-time” means energy delivery can be adjusted continuously (e.g., at least once every second) during the energy delivery, as the second skin property is continuously measured.

In Step (132), energy delivery is paused when the second skin property reaches a predetermined threshold value. For example, the treatment is paused when the temperature of the skin area receiving energy reaches 45° C., so that the skin is not overheated or burned. In another example, the treatment is paused when the neuron discharging rate or nerve conduction velocity of the skin area is increased or decreased by 10%.

In Step (133), energy delivery is resumed, when the second skin property reaches a predetermined threshold value. For example, the treatment is resumed when the temperature drops below 40° C., or when neuron discharging rate or the nerve conduction velocity returns to baseline level before the treatment.

In Step (140), treatment is terminated when the amount of energy is delivered, or when a second skin property fails to return to a predetermined level. For example, if neuron discharging rate or the nerve conduction velocity fails to returns to baseline level within 5 minutes after pausing energy delivery, the treatment is terminated.

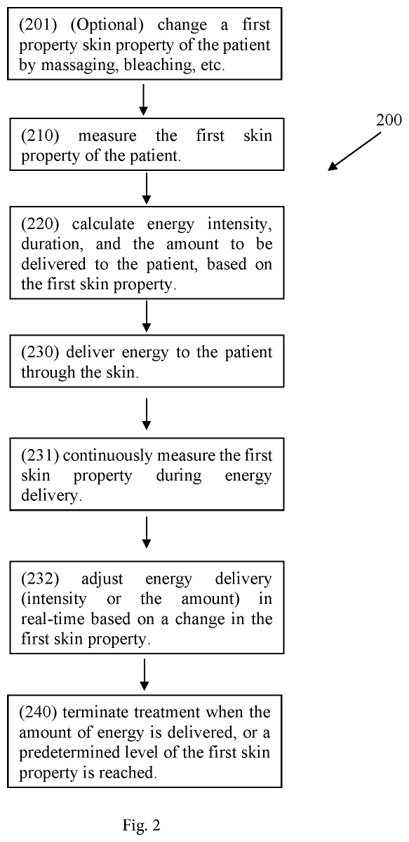

The flowchart 200 in FIG. 2 is similar to the flowchart 100 in FIG. 2, but with some variations. In Step 231, the first skin property is continuously or periodically measured during energy delivery. In Step (232), energy delivery (intensity or the amount) is adjusted in real-time based on a change in the first skin property. As used herein, “real-time” means energy delivery can be adjusted continuously (e.g., at least once every second) during the energy delivery, as the first skin property is continuously measured.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A method of monitoring stem cell activation in a patient, comprising:
measuring a first skin property of the patient;
calculating an amount of energy to be delivered to the patient based on the first skin property;
delivering energy to the patient through at least a portion of the patient's skin;
continuously or periodically measuring a second skin property during energy delivery;
pausing delivery when the second skin property reaches a first predetermined threshold value;
resuming delivery when the second skin property reaches a second predetermined threshold value; and
terminating delivery of energy when the amount of energy is delivered.

2. The method of claim 1, wherein measuring the first skin property of the patient comprises characterizing light reflection from the patient's skin.

3. The method of claim 1, wherein the first skin property is selected from the group consisting of coloration, skin texture, thickness, and pigmentation.

4. The method of claim 1, wherein the second skin property is selected from the group consisting of moisture, permeability, temperature, blood circulation in skin tissue, skin conductivity, cutaneous blood pressure, and skin metabolism.

5. The method of claim 1, wherein the second skin property comprises a nerve activity of the patient.

6. The method of claim 5, wherein the nerve activity comprises a discharge rate.

7. The method of claim 5, wherein the nerve activity comprises a nerve conductivity.

8. The method of claim 5, further comprising adjusting the amount of energy to be delivered based on a changed in the second skin property measured during energy delivery.

9. The method of claim 1, wherein calculating energy intensity is based on an empirical correlation between the first skin property and the amount of energy required to sufficiently activate stem cells.

10. The method of claim 1, the energy delivery is paused when a temperature of the skin surface reaches 45° C.

11. The method of claim 1, further comprising massaging the patient's skin before delivery of the energy to the patient.

12. The method of claim 1, further comprising bleaching the patient's skin before delivery of the energy to the patient.

13. A method of monitoring stem cell activation in a patient, comprising:
changing a first property skin property of the patient;
measuring the first skin property of the patient;
calculating an amount of energy and an intensity of energy to be delivered to the patient based on the first skin property;
delivering energy to the patient through at least a portion of the patient's skin;
continuously measuring the first skin property during energy delivery;
adjusting the intensity of energy delivery in real-time based on a change in the first skin property;
terminating delivery of energy when the amount of energy is delivered.

14. The method of claim 13, wherein changing the first property skin property of the patient comprising treating the patient's skin with a chemical formulation comprising carboxymethylcellulose.

15. The method of claim 13, wherein the first skin property is selected from the group consisting of coloration, skin texture, thickness, and pigmentation.

16. A method of monitoring stem cell activation in a patient, comprising:
delivering energy to the patient through at least a portion of the patient's skin;
noninvasively characterizing a biomolecule of the patient;
pausing or terminating delivery when the characterization of the biomolecule reaches a first predetermined threshold value.

17. The method of claim 16, wherein the noninvasive characterization comprises determining composition of a body fluid.

18. The method of claim 16, wherein the biomolecule is a biomarker of stem cell activation.

19. The method of claim 16, wherein the biomolecule is a biomarker of skin damage.

20. The method of claim 16, wherein the biomolecule is a reactive oxygen species.
